(19) United States
(12) Patent Application Publication
(10) Pub. No.: US 2022/0251502 A1
(43) Pub. Date: Aug. 11, 2022

(54) METHODS FOR REDUCING THE OXIDATION LEVEL OF CYSTEINE RESIDUES IN A SECRETED RECOMBINANTLY-EXPRESSED PROTEIN DURING CELL CULTURE

(71) Applicant: NOVARTIS AG, Basel (CH)

(72) Inventors: Nuno BUXO CARINHAS, Basel (CH); Huanchun CUI, Lexington, MA (US); David GARCIA, Saint Louis (FR); Mathias GOEBEL, Freiburg (DE); Joseph SHULTZ, Nahant, MA (US)

(21) Appl. No.: 17/557,921

(22) Filed: Dec. 21, 2021

Related U.S. Application Data
(60) Provisional application No. 63/129,091, filed on Dec. 22, 2020.

(51) Int. Cl. C12N 5/00; C12P 21/00

(52) U.S. Cl. CPC C12N 5/0018; C12N 2500/32; C12P 21/005

(57) ABSTRACT

The present disclosure relates to methods for reducing the oxidation level of cysteine residues in recombinant polypeptides such as anti-IL-17 antibodies during cell culture (e.g., a preparation of secukinumab antibodies) that have been recombinantly produced by mammalian cells. Also provided are purified preparations of recombinant polypeptides such as anti-IL-17 antibodies or antigen binding fragments thereof produced by such methods, e.g, purified preparations of secukinumab. Also provided are purified preparations of recombinant polypeptides produced by such methods wherein the level of active recombinant polypeptide in the preparation is high.

### TECHNICAL FIELD

The present disclosure relates to methods for reducing the oxidation level of one or more cysteine residues in a secreted recombinantly expressed protein during cell culture, e.g. during recombinant production of an anti-IL-17 antibody such as secukinumab by mammalian cells.

### BACKGROUND OF THE DISCLOSURE

Classical antibodies are composed of two light chains (L) with a molecular weight of about 25 kD each and two heavy chains (H) with a molecular weight of about 50 kD each. The light and heavy chains are connected by a disulfide bond (L-S-S-H) and the two LH units are further linked between the heavy chains by two disulfide bonds. The general formula of a classical antibody is L-SS-H(-SS-)₂H-SS-L or simply H₂L₂ (HHLL). Besides these conserved inter-chain disulfide bonds, there are also conserved intra-chain disulfide bonds. Both types of disulfide bonds are important for the stability and behavior (e.g., affinity) of an antibody. Generally, a disulfide bond is produced by two cysteine residues (Cys-SH) found at conserved positions in the antibody chains, which spontaneously form the disulfide bond (Cys-S-S-Cys). Disulfide bond formation is determined by the redox potential of the environment and by the presence of enzymes specialized in thiol-disulfide exchange. The internal disulfide bonds (Cys-S-S-Cys) stabilize the three-dimensional structure of an antibody.

There are antibodies that contain an additional free cysteine(s) (i.e., unpaired cysteine). In some cases, one or more free cysteine is involved in antigen recognition and binding, e.g., because the free cysteine is present in a complementarity determining region of the antibody. For these antibodies, modification of a free cysteine can have a negative effect on the activity and stability of the molecule, and can lead to increased immunogenicity. As a result, processing of these antibodies can be difficult, as the end product may contain a substantial amount of inactive, misfolded and/or useless antibody material. US20090280131, which is incorporated by reference herein in its entirety, provides anti-IL-17 antibodies, e.g., secukinumab (i.e., AIN457) with a free cysteine residue after the cis-proline in the light chain complementarity determining region (CDR) 3 loop (L-CDR3) (i.e., amino acid eight of L-CDR3 as set forth as SEQ ID NO:6, which corresponds to amino acid 97 of the light chain variable region as set forth as SEQ ID NO:10, herein after referred to as “CysL97”). In order to maintain full activity, the unpaired cysteine residue of secukinumab cannot be masked by oxidative disulfide pairing with other cysteine residues or by oxidation with exogenous compounds (e.g., formation of mixed disulfides with other proteins, derivatization with cell metabolites (e.g., cysteine or glutathione), and formation of sulfoxides by oxygen). Unfortunately, because secukinumab is manufactured using mammalian cells, which secrete the secukinumab into the cell culture media, undesired cell-based modifications of CysL97 do occur.

Similarly, engineering of a free cysteine into an antibody sequence can be useful for facilitating site directed conjugation of a chemical linker, drug, label, and/or other moiety. For example, Junutula et al. (Nat. Biotechnol., 2008, 26, 925-932) introduced an engineered cysteine into an anti-MUC16 antibody by mutation of heavy chain alanine 114. The authors found that expression of the mutated antibodies in Chinese Hamster Ovary (CHO) cells generated an antibody with the engineered cysteine residue capped as a disulfide with cysteine or glutathione. Thus, the engineered cysteine reside must be treated to remove the undesired cell-based modification.

Methods for selectively reducing antibodies having oxidation of a free cysteine reside have been reported. For example, reduction of oxidized CysL97 in a preparation of IL-17 antibodies that have been recombinantly produced by mammalian cells are disclosed in WO2016/103146A1. Specifically, downstream processing steps are applied, such as contacting a preparation comprising an antibody with at least one reducing agent in a system to form a reducing mixture; and incubating the reducing mixture while maintaining a volumetric oxygen mass-transfer coefficient (kLa*) in the system of <about 0.37 h-1, said kLa* being calculated by adapting a dissolved oxygen curve to a saturation curve. Similarly, Junutula et al. reported a procedure employing a strong reducing agent (e.g., tris(2carboxyethyl)phosphine [TCEP] or dithiothreitol [DTT])), purification of the reduced antibody, and subsequent reoxidation of interchain disulfide bonds using Cu²⁺ or dehydro-ascorbic acid.

However, such downstream method steps may require expensive equipment, additional purification steps, and result in prolonged process lead times. Thus, there is a need for improved processes allowing faster and/or less costly total manufacturing. As such, methods in the upstream processing steps of recombinant antibody production can also be evaluated for optimization.

The culture of secreted mammalian cells for industrial applications, such as expression of recombinant polypeptides, requires media that support growth and production. Such media must support high viable cell densities while also stimulating the synthesis and extracellular transport of biologic products. Early media development efforts yielded basic formulations to sustain growth, viability, and cellular function, albeit comprising animal sourced components, and complex constituents used in batch culture mode. Subsequent improvements included the development of serum-free and chemically defined (CD) media, the identification of critical nutrients, growth factors, and potentially inhibitory or toxic cellular metabolites, and the use of fed-batch and perfusion culture techniques to optimize nutrient delivery while minimizing accumulation of unwanted waste products.

All cell culture media require similar basic nutrients to support cellular growth. Amino acids are key components in cell culture media and studies have shown that small changes in amino acid composition of cell culture media can alter growth profiles and titers. For example, Ghaffari et al. (Biotechnol Progress. 2020; 36:e2946) report that maintaining availability of the so-called non-essential amino acid cysteine is a critical process parameter for high yield recombinant protein production in common CHO cell lines. However, cysteine is easily oxidized at the pH and oxygen and metal rich conditions of typical cell culture media. However, cysteine can also facilitate undesired oxidation of free cysteine residues of a recombinant protein. Therefore, a cysteine feeding strategy typically requires a high degree of optimization in order to achieve high yields of a recombinantly expressed protein from CHO cells.

Many cell culture media and media feeds are available commercially to supply key nutrients. However to optimize quality and yield of a secreted recombinant polypeptide having a reduced cysteine residue under production, there is still a need to tailor the media and feeds to the recombinant polypeptide. With many parameters that can altered in the cell culture conditions, process optimization is complex and even for commercially available recombinant polypeptides such as antibodies, there is still a need to provide optimized processes for industrial production.

### SUMMARY OF THE DISCLOSURE

Despite extensive research and optimization in the field of cell culture media, it has been surprisingly found that reducing the amount of added cysteine in a mammalian cell culture medium, to reduce the concentration of cys equivalents in the culture medium can reduce undesired modification of a free cysteine of an expressed antibody. Such a reduced amount of undesired modification of the free cysteine can result in improved antibody activity and/or avoid the need for subsequent reduction and optionally re-oxidation of the antibody.

According to a first aspect, the present disclosure provides a process for the production of a recombinant polypeptide in a fed batch cell culture, comprising the steps of:

a. culturing mammalian cells in a cell culture medium comprising a base medium and one or more feed media, wherein the base medium comprises a concentration of cys equivalents of about 0.3 g/L and wherein the feed media comprises a concentration of cys equivalents of less than about 0.8 g/L, and wherein the concentration of cumulative cys equivalents in the cell culture media are less than about 0.4 g/L;

b. expressing the recombinant polypeptide and

c. recovering the polypeptide from the culture medium.

The cumulative cys equivalents in the cell culture medium are the total concentration of cysteine and cystine in the cell culture medium derived from the base medium and/or the feed media. The cumulative cys equivalents may be at a different concentration at the start of a fed batch process compared to at the end of the process, for example, after a period of hours or days, and may vary during the process when the feed media are added to the cell culture medium. In one embodiment, the concentration of cys equivalents in the cell culture medium at the start of a fed batch process may be less than about 0.6 g/L. For example, less than about 0.5 g/L, less than about 0.4 g/L and preferably less than about 0.3 g/L. During the fed batch process, the concentration of cys equivalents may vary due to the addition of cys equivalents (contributed either by the addition of cysteine or cystine) to the cell culture medium of between about 0.3 g/L to about 0.8 g/L. To give this concentration of cys equivalents, the concentration of cysteine added to the cell culture medium in the feed media can be less than about 1 g/L. For example, the base medium can comprise no added cysteine and the feed media can comprise a concentration of cysteine of less than about 1.0 g/L, for example, less than about 0.9 g/L, less than about 0.8 g/L, and preferably less than about 0.7 g/L. In one embodiment, the base medium can comprise no added cysteine and the feed media can comprise a concentration of cysteine of about 0.66 g/L. In another embodiment, the base medium can comprise no added cysteine and the feed media can comprise a concentration of cysteine of about 0.33 g/L. In a further embodiment, the base medium can comprise no added cysteine and the feed media can also comprise no cysteine.

At the end of a fed batch process as described herein, the concentration of cumulative cys equivalents in the cell culture media can be less than about 0.4 g/L. In a standard fed batch cell culture in which no change has been made to reduce the cys equivalents in the base medium and/or feed media, the concentration of cumulative cys equivalents in the cell culture media can be about 0.6 g/L.

In one embodiment, the recombinant polypeptide produced in the fed batch cell culture is an antibody, preferably the anti-IL-17 antibody secukinumab.

In one embodiment, the mammalian cells used in the red batch cell culture are selected from the group consisting of: CHO cells, HEK cells and SP2/0 cells. For example, the mammalian cells can be CHO cells, selected from the group consisting of: CHO-S, CHO Kl , CHO pro3-, CHO DG44, CHO P12, or the dhfr- CHO cell line DUK-BII, DUXBI 1 or CHO-K1SV.

In a second aspect, the process described above to reduce the concentration of cys equivalents in the cell culture medium can be combined with a downstream processing step of selective reduction, wherein the antibody is incubated with at least one reducing agent in a system to form a reducing mixture and incubating the reducing mixture while maintaining a volumetric oxygen mass-transfer coefficient (kLa*) in the system of <about 0.37 h-1, said kLa* being calculated by adapting a dissolved oxygen curve to a saturation curve. Preferably the antibody is secukinumab. Such a downstream processing step for selectively reducing the cysteine residue at position CysL97 in a preparation of IL-17 antibodies is disclosed in WO2016/103146A1.

The recombinant polypeptide produced according to the process of the present disclosure can be prepared for administration to a human patient by carrying out further steps to prepare a pharmaceutical product. For example, where the recombinant polypeptide is an antibody, it is necessary to purify the antibody and formulate the antibody with various excipients to provide a pharmaceutical composition suitable for administration to the patient. In addition, the antibody can be packaged with a leaflet comprising instructions for administration to the patient. Such a leaflet may provide the dose, route of administration, regimen, and total treatment duration for use with the enclosed antibody.

In one aspect, the present invention provides a process for production of a recombinant polypeptide by mammalian cell culture, comprising the steps of: a) culturing mammalian cells (e.g., selected from the group consisting of CHO cells, HEK cells and SP2/0 cells) in a culture comprising a cell culture medium, wherein the cell culture medium comprises a reduced concentration of cys equivalents as compared to a control base medium; b) exchanging all or a portion of the cell culture medium in the culture with fresh cell culture medium by perfusion, wherein the fresh cell culture medium comprises a reduced concentration of cys equivalents as compared to a control exchange medium; c) expressing the recombinant polypeptide and d) recovering the polypeptide from the culture.

In some embodiments, the perfusion cell culture medium comprises a concentration of cys equivalents of from about 0.1 g/L to less than about 0.6 g/L, from about 0.2 g/L to less than about 0.5 g/L, from about 0.25 g/L to less than about 0.4 g/L, or from 0.3 g/L to about 0.4 g/L. In some embodiments, the fresh perfusion cell culture medium comprises a concentration of cys equivalents of from about 0.1 g/L to less than about 1.1 g/L, from about 0.2 g/L to less than about 0.9 g/L, from about 0.25 g/L to less than about 0.6 g/L, or from 0.3 g/L to about 0.4 g/L. In some embodiments, the culture medium comprises a concentration of cys equivalents of about 0.3 g/L. In some embodiments, the fresh culture medium comprises a concentration of cys equivalents of about 0.3 g/L.

In some embodiments, the cumulative cys equivalents added to the perfusion culture are less than about 11 g/L, less than about 9 g/L, or less than about 7 g/L. In some embodiments, the cumulative cys equivalents added to the culture are from about 3 g/L to less than about 11 g/L, from about 4 g/L to less than about 11 g/L, from about 5 g/L to less than about 11 g/L, from about 3 g/L to less than about 9 g/L, from about 4 g/L to less than about 9 g/L, from about 5 g/L to less than about 9 g/L, or preferably from about 5 g/L to less than about 7 g/L.

In some embodiments the cumulative cys equivalents added to the perfusion culture are less than about 1 g/L per day, less than 0.9 g/L per day, less than 0.7 g/L per day, less than 0.6 g/L per day, less than about 0.5 g/L per day, less than about 0.4 g/L per day, or less than about 0.3 g/L per day. In some embodiments, the cumulative cys equivalents added to the perfusion culture are from about 0.1 g/L per day to less than about 1 g/L per day, preferably from about 0.2 g/L per day to less than about 0.6 g/L per day, more preferably from about 0.2 g/L per day to less than 0.5 g/L per day, or about 0.3 or 0.4 g/L per day.

### BRIEF DESCRIPTION OF THE FIGURES

FIG. 1 is a graph showing activity of an antibody sample according to an embodiment.

FIG. 2 shows the effect on antibody activity (%) by reducing the cysteine/cystine concentration (cys equivalents) in variations of a perfusion media over time in days (x-axis). The greater reduction in cysteine/cystine concentration, results in greater retention of antibody activity compared to the baseline of perfusion media (y-axis).

FIG. 3 shows that reducing the amount of cysteine in the base medium and/or feed media has little effect on the concentration of secukinumab (mg/ml; y-axis) produced over time (days; x-axis).

FIG. 4 shows the end culture concentrations of secukinumab (mg/ml) at day 12 vary little between baseline (cell culture medium comprising standard base and feed media) and the three Variant base media and/or feed media tested.

FIG. 5 shows the activity of antibody samples (%) produced using the three Variant base media and/or feed media tested, compared to baseline (cell culture medium comprising standard base medium and feed media).

FIG. 6 shows the activity of antibody samples (%) produced by perfusion culture using test perfusion medium having no cysteine in the perfusion medium and therefore 50% reduced cys equivalents compared to control perfusion cell culture medium comprising a normal level of cysteine and cys equivalents. The cumulative cys equivalents added to test and control perfusion cell culture, with approximately one complete media exchange per day, was approximately 5.875 g/L (˜0.31 g/L/day) and 11.642 g/L (0.61 g/L/day) respectively.

### DETAILED DESCRIPTION OF THE DISCLOSURE

It is an object of the disclosure to provide methods for reducing the oxidation level of cysteine residues in recombinant polypeptides such as anti-IL-17 antibodies during cell culture, e.g. during recombinant production of secukinumab by mammalian cells.

The term “comprising” encompasses “including” as well as “consisting” e.g. a composition “comprising” X may consist exclusively of X or may include something additional e.g. X+Y.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The relative molecular mass of secukinumab, based on post-translational amino acid sequence, is 147,944 Daltons. This molecular weight (i.e., 147,944 Daltons) is used in the calculation of secukinumab molarity values and molar ratios throughout the instant disclosure. However, during production in CHO cells, a C-terminal lysine is commonly removed from each heavy chain. The relative molecular mass of secukinumab lacking a C-terminal lysine from each heavy chain is 147,688 Daltons. A preparation of secukinumab contains a mixture of molecules with and without C-terminal lysine residues on the heavy chain. The secukinumab molarity values (and ratios employing these molarity values) used in the instant disclosure are therefore estimates, and the term “about”, “approximate” and the like in reference to these numerical values encompasses at least this variation in relative molecular mass and the resulting calculations made therewith.

The word “substantially” does not exclude “completely” e.g. a composition which is “substantially free” from Y may be completely free from Y. Where necessary, the word “substantially” may be omitted from the definition of the disclosure.

Large scale cultivation of cells can be used for instance by the various fermentation processes established in industrial biotechnology. Discontinuous and continuous cell culture processes, like perfusion and chemostat, can be utilized using the cell culture media according to the present invention. Discontinuous processes, including repeated fed-batch and repeated batch, are one preferred embodiment. Generally, the methods and compositions of the present invention are directed to the production of a secreted polypeptide by cell culture.

The batch cell culture includes fed-batch culture or simple batch culture. The term “fed batch cell culture” refers to cell culture wherein cells and cell culture medium are supplied to the culturing vessel initially and additional culture nutrients are fed continuously or in discrete increments to the culture during the culturing process with or without periodic cell and/or product harvest before termination of the culture. The term “simple batch culture” relates to a procedure in which all components for cell culturing including the cells and the cell culture medium are supplied to the culturing vessel at the start of the culturing process. Preferably, the cells cultivated in the cell culture medium according to the present invention are CHO cells.

The term “cell culture medium” refers to an aqueous solution of nutrients which can be used for growing cells over a prolonged period of time. Typically, cell culture media include the following components: a source of energy, which will be usually a carbohydrate compound, preferably glucose, amino acids, preferably the basic set of amino acids, including all essential and non-essential amino acids, vitamins and/or other organic compounds which are required at low concentrations, free fatty acids, and inorganic compounds including trace elements, inorganic salts, buffering compounds and nucleosides and bases.

The term “growth medium” refers to a cell culture medium which is normally used during expansion phase of an overall production process. The expansion phase is the first period of the overall cultivation/production process which is predominantly characterized by high cell growth and less polypeptide production. The expansion phase serves the purpose of expanding the cells, which means generating an adequate number of cells which are in the exponential growth phase to inoculate a production bioreactor.

The term “production medium” refers to a cell culture medium which is normally used during production phase of the overall production process. The production phase is a second phase of the overall cultivation/production process which serves the purpose of producing high amounts of product. During the production phase the cells should be maintained in viable and productive mode as long as possible.

The use of cell culture media in the field of pharmaceutical industry, for instance for the production of therapeutically active recombinant polypeptides, does generally not allow the use of any material of animal origin due to safety and contamination issues. Therefore, the cell culture medium according to the present invention is preferably a serum- and/or protein-free medium. The term “serum- and/or protein-free medium” represents a fully chemically defined medium, containing no additives from animal source like tissue hydrolysates, fetal bovine serum or the like. Further, proteins, especially growth factors like insulin, transferrin or the like are also preferably not added to the cell culture according to the present invention. Preferably, the cell culture medium according to the present invention is also not supplemented with a hydrolysed protein source like soybean, wheat or rice peptone or yeast hydrolysate or the like.

The term “base medium”, is a medium used for culturing cells which is, itself, directly used to culture the cells and is not used as an additive to other media, although various components may be added to a base medium. For example, if CHO cells were cultured in DMEM, a well-known, commercially-available medium for mammalian cells, and periodically fed with glucose or other nutrients, DMEM would be considered the base medium. A “feed medium” is a medium used as a feed in a cell culture, which may be a fed batch cell culture. A feed medium, like a base medium, is designed based on the needs of the particular cells being cultured and a feed medium can have higher concentrations of most, but not all, components of a base culture medium. For example, some components, such as, for example, nutrients including amino acids or carbohydrates, may be at about 5, 6, 7, 8, 9, 10, 20, 50, 100, 200, 400, 600, 800, or even about 1000 times of their normal concentrations in a base medium. Some components, such as salts, maybe kept at about the same concentration of the base medium concentration, to keep the feed isotonic with the base medium. Some components are added to keep the feed physiologic, and some are added because they are replenishing nutrients to the culture.

The cell culture medium according to the present invention can be used in various cell culture processes. Cultivation of cells can be carried out in adherent culture, for instance in monolayer culture or preferably in suspension culture.

The polypeptides that can be produced from the cell cultures and the cell culture media according to the present invention are not limited. The polypeptides can be recombinant or not recombinant. The term “polypeptide” as used herein encompasses molecules composed of a chain of more than two amino acids joined by peptide bonds; molecules containing two or more such chains; molecules comprising one or more such chains being additionally modified, e.g. by glycosylation. The polypeptide can contain one or more native disulfide bonds. The polypeptide can contain a native or engineered free cysteine. The term polypeptide is intended to encompass proteins.

The preferred class of polypeptides produced by cell cultures and the cell culture media according to the present invention are recombinant antibodies.

The term “antibody” as referred to herein includes whole antibodies and any antigen-binding portion or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

The term “antigen-binding fragment” of an antibody as used herein, refers to fragments of an antibody that retain the ability to specifically bind to an antigen (e.g., IL-17). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Exemplary antigen binding sites include the CDRs of secukinumab as set forth in SEQ ID NOs:1-6 and 11-13 (Table 1), preferably the heavy chain CDR3. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., (1988) Science, 242: 423-426; Huston et al., (1988) Proc. Natl. Acad. Sci., 85: 5879-5883). Such single chain antibodies are also intended to be encompassed within the term “antibody”. Single chain antibodies and antigen-binding portions are obtained using conventional techniques known to those of skill in the art.

An “isolated antibody”, as used herein, refers to an antibody that is substantially free of other antibodies having different antigenic specificities (e.g., an isolated antibody that specifically binds IL-17 is substantially free of antibodies that specifically bind antigens other than IL-17). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo). In some embodiments of the disclosed processes and compositions, the IL-17 antibody is a human antibody, an isolated antibody, and/or a monoclonal antibody.

The term “IL-17” refers to IL-17A, formerly known as CTLA8, and includes wild-type IL-17A from various species (e.g., human, mouse, and monkey), polymorphic variants of IL-17A, and functional equivalents of IL-17A. Functional equivalents of IL-17A according to the present disclosure preferably have at least about 65%, 75%, 85%, 95%, 96%, 97%, 98%, or even 99% overall sequence identity with a wild-type IL-17A (e.g., human IL-17A), and substantially retain the ability to induce IL-6 production by human dermal fibroblasts.

The term “K_{D}” is intended to refer to the dissociation rate of a particular antibody-antigen interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system. In some embodiments, the IL-17 antibody or antigen binding fragment, e.g., secukinumab, has a K_{D} of about 100-250 pM for humanlL-17.

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IL-17 of various species are known in the art, including for example, ELISAs, western blots and RIAs. The binding kinetics (e.g., binding affinity) of the antibodies also can be assessed by standard assays known in the art, such as by Biacore analysis.

An antibody that “inhibits” one or more of these IL-17 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (or when a control antibody of irrelevant specificity is present). An antibody that inhibits IL-17 activity affects a statistically significant decrease, e.g., by at least about 10% of the measured parameter, by at least 50%, 80% or 90%, and in certain embodiments of the disclosed methods and compositions, the IL-17 antibody used may inhibit greater than 95%, 98% or 99% of IL-17 functional activity.

The term “derivative”, unless otherwise indicated, is used to define amino acid sequence variants, and covalent modifications (e.g., pegylation, deamidation, hydroxylation, phosphorylation, methylation, etc.) of an IL-17 antibody or antigen binding fragment thereof, e.g., secukinumab, according to the present disclosure, e.g., of a specified sequence (e.g., a variable domain). A “functional derivative” includes a molecule having a qualitative biological activity in common with the disclosed IL-17 antibodies. A functional derivative includes fragments and peptide analogs of an IL-17 antibody as disclosed herein. Fragments comprise regions within the sequence of a polypeptide according to the present disclosure, e.g., of a specified sequence. Functional derivatives of the IL-17 antibodies disclosed herein (e.g., functional derivatives of secukinumab) preferably comprise V_{H} and/or V_{L} domains having at least about 65%, 75%, 85%, 95%, 96%, 97%, 98%, or 99% overall sequence identity with the V_{H} and/or V_{L} sequences of the IL-17 antibodies and antigen binding fragments thereof disclosed herein (e.g., the V_{H} and/or V_{L} sequences of Table 1), and substantially retain the ability to bind human IL-17 or, e.g., inhibit IL-6 production of IL-17 induced human dermal fibroblasts.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative IL-17 antibody (e.g., a derivative of secukinumab, e.g., a secukinumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are well known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

“Amino acid(s)” refer to all naturally occurring L-a-amino acids, e.g., and include D-amino acids. The phrase “amino acid sequence variant” refers to molecules with some differences in their amino acid sequences as compared to the sequences according to the present disclosure. Amino acid sequence variants of an antibody according to the present disclosure, e.g., of a specified sequence, still have the ability to bind the human IL-17 or, e.g., inhibit IL-6 production of IL-17 induced human dermal fibroblasts. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The phrases “free cysteine”, “non-traditional cysteine” and “unpaired cysteine” interchangeably refer to a cysteine that is not involved in conserved antibody disulfide bonding or in reference to a non-antibody polypeptide a cysteine that does not form a disulfide bond with another unpaired cysteine in the wild-type structure of the polypeptide. The free cysteine may be present in an antibody framework region or a variable region (e.g., within a CDR). In secukinumab, amino acid eight of L-CDR3 as set forth as SEQ ID NO:6, which corresponds to amino acid 97 of the light chain variable region as set forth as SEQ ID NO:10 (herein after referred to as CysL97) is a free cysteine. Each molecule of secukinumab comprises two such free cysteine residues—one in each V_{L} domain.

The term “selective reduction” as used herein refers to a method for selectively reducing CysL97 in a preparation of IL-17 antibodies that have been recombinantly produced by mammalian cells as disclosed in WO2016/103146A1. Specifically, downstream processing steps are applied, such as contacting a preparation comprising an antibody with at least one reducing agent in a system to form a reducing mixture; and incubating the reducing mixture while maintaining a volumetric oxygen mass-transfer coefficient (kLa*) in the system of <about 0.37 h-1, said kLa* being calculated by adapting a dissolved oxygen curve to a saturation curve.

During recombinant polypeptide expression in a cell culture system, cysteine can be included in culture media or, for example, during a fed-batch process included in the base medium and/or added to the culturing vessel in a media feed. Cysteine refers to L-cysteine rather than D-cysteine and can be added in the form of a salt such as cysteine hydrochloride monohydrate. Typically, monomeric cysteine dimerizes immediately when added to a cell culture media and therefore exists only in the dimeric form of cystine. This redox reactions leads to the formation of a disulfide bond between the 2 monomeric cysteine molecules. The concentration of cysteine in a base or feed medium of the invention can be less than about 5.0, 4.0, 3.0, 2.5, 2.0, 1.5, 1.0, 0.90, 0.80, 0.70, 0.60, 0.50, 0.45, 0.40, 0.35, 0.30, 0.25, 0.20, 0.15 or 0.10 g/L. Alternatively, the base medium and/or feed media can be free of cysteine.

Cystine can also be present in the base cell culture media or added to a culture media as a media feed, for example, as part of a tyrosine cystine stock solution. The concentration of cysteine in a base or feed media of the invention can be less than about 5.0, 4.0, 3.0, 2.5, 2.0, 1.5, 1.0, 0.90, 0.80, 0.70, 0.60, 0.50, 0.45, 0.40, 0.35, 0.30, 0.25, 0.20, 0.15 or 0.10 g/L. Alternatively, the base medium and/or feed medium can be free of cysteine.

Although cysteine added to a culture medium usually oxidizes to form cystine, some of the cystine added to a medium may be reduced to form cysteine, and therefore the numbers given above for these concentrations refer to the concentration of cysteine or cystine which is actually added to the medium without later determination of what proportion of this may have been oxidized or reduced. Therefore, in the context of the amounts of cysteine and/or cystine available to cells in the culture vessel, the term “cys-equivalents” can be used. This term, as used herein, refers to the amount or concentration of cysteine and cystine in a cell culture media or media feed in total. Cys-equivalents will be the cysteine/cystine available to the cells in the cell culture medium, in the culture vessel whether derived from the base medium and/or feed media. Therefore in a culture vessel, for example, in the cell culture medium for a fed batch process, the concentration of total cys equivalents can be less than about 5.0, 4.0, 3.0, 2.5, 2.0, 1.5, 1.0, 0.90, 0.80, 0.75, 0.70, 0.65, 0.60, 0.55, 0.50, 0.45, 0.40, 0.35, 0.30, 0.25, 0.20, 0.15 or 0.10 g/L.

Since the cys equivalents in a cell culture medium, particularly in a fed batch process can be derived from the base medium and/or the feed media, the term “cumulative cys equivalents” is used to refer to the total amount or concentration of cys equivalents in the cell culture medium derived from the base medium and/or the feed media. The cumulative cys equivalents may be at a different concentration at the start of a fed batch process compared to at the end of the process, for example, after 5 days, 8 days, 10 days, 11 days or 12 days, and may vary during the process, when the feed media are added to the cell culture medium. In a fed batch cell culture process under standard culture conditions, the concentration of cys equivalents in the cell culture media can be in the range of about 0.5 g/L to about 0.6 g/L or about 4 mM to about 5 mM. In a process according to the present disclosure, the concentration of cys equivalents in the cell culture medium at the start of a fed batch process may be less than about 0.6 g/L or less than about 4.5 mM. For example, less than about 0.5 g/L, less than about 0.4 g/L and preferably less than about 0.3 g/L, or less than about 3.5 mM, less than about 3.0 mM and preferably less than about 2.5 mM. During the fed batch process, the concentration of cys equivalents may vary due to the addition of cys equivalents (contributed either by the addition of cysteine or cystine, or both) to the cell culture medium of between about 0.3 g/L to about 0.8 g/L. To achieve this concentration of cys equivalents, the concentration of cysteine added to the cell culture medium in the feed media can be less than about 1 g/L. For example, the base medium can comprise no added cysteine and the feed media can comprise a concentration of cysteine of less than about 1.0 g/L, for example, less than about 0.9 g/L, less than about 0.8 g/L, and preferably less than about 0.7 g/L. In one embodiment, the base medium comprises no added cysteine and the feed media comprise a concentration of cysteine of about 0.66 g/L. In another embodiment, the base medium comprises no added cysteine and the feed media comprise a concentration of cysteine of about 0.33 g/L. In a further embodiment, the base medium comprises no added cysteine and the feed media comprise no cysteine.

The feed media can be added to the fed batch cell culture at various time points over the course of the culture period. For example the feed media can be added to the cell culture medium on a daily basis during the culture period or can be added after an initial period of two or three days and then added on a daily basis. The feed media can be added once, twice, three times, four times, five times, six times, seven times etc, during the fed batch cell culture process.

At the end of a fed batch cell culture process as described herein, the concentration of cumulative cys equivalents in the cell culture media can be less than about 0.4 g/L or less than about 3 mM. In a standard fed batch cell culture in which no change has been made to reduce the cys equivalents in the base medium and/or feed media, the concentration of cumulative cys equivalents in the cell culture media can be about 0.6 g/L or about 5 mM. This concentration of cumulative cys equivalents in a standard, e.g., fed batch, cell culture medium can also be referred to as baseline.

In a perfusion culture fresh cell culture media can be added to the cell culture by perfusion mediated media exchange. The exchange can be continuous or discontinuous (e.g., performed at various time points over the course of the culture period). For example fresh media can be exchanged with the cell culture medium in the culture on a continuous basis during the culture period or perfusion exchange can be initiated after an initial period of two or three days and then exchanged continuously. In some embodiments, the perfusion is performed under conditions sufficient to replace at least 50%, preferably 75%, more preferably 99% or about 100% of the cell culture media per day of culture.

Accordingly, the total volume of media consumed during a perfusion batch culture can be much higher than a fed batch culture. Thus, the cumulative cys equivalents added to the perfusion culture can be correspondingly higher, while still achieving the lower level of oxidation of free cysteine oxidation provided by the methods and compositions described herein. For example, a 1000 L perfusion batch culture having approximately 100% media exchange per day can be cultured for 19 days, for a total volume of consumed media of approximately 19,000 L. However, the total culture volume can remain approximately 1000 L. In such an embodiment, the cumulative cys equivalents added to the control perfusion culture can be greater than about 7 g per liter of culture volume, greater than about 8 g/L, greater than about 10 g/L, or greater than about 11 g/L, or about 11 g/L. In some embodiments, the cumulative cys equivalents added to the control perfusion culture can be, or be about, 11 or 11.5 g/L. Similarly, the cumulative cys equivalents added to a 19 day 1000 L perfusion culture with an exchange of about 100% of media per day and cultured under reduced cysteine and/or reduced cys equivalents conditions can be less than about 7 g per liter of culture volume, 6.5 g/L, 6 g/L, or about 5.8 g/L cumulative cys equivalents.

Thus in reference to a perfusion batch culture, a reduced cys equivalents condition can be characterized by a reduced cumulative cys equivalents condition that is normalized by the number of culturing days. Thus for example, where a 19 day perfusion culture is cultured with approximately 1 complete exchange of media volume per day under control conditions of about 0.6 g/L cys equivalents in the starting and exchange media, a normalized cumulative cys equivalents can be about 0.6 g/L/day. Similarly, where a 19 day perfusion culture is cultured with approximately 1 complete exchange of media volume per day under test conditions of about 0.3 g/L cys equivalents in the starting and exchange media (or less), a normalized cumulative cys equivalents can be less than 0.6 g/L/day, such as about 0.3 g/L/day.

In some embodiments, a secreted recombinant protein having a free cysteine is produced in a perfusion batch process by culturing mammalian cells in base perfusion media containing less than 0.62 g/L cys equivalents and adding to the culture less than 0.62 g/L cys equivalents per day by continuously or discontinuously exchanging (e.g., by perfusion) all or a portion of the cell culture media with a perfusion exchange media. In some cases, the process comprises adding from about 0.2 g/L cys equivalents to less than 1 g/L cys equivalents to the culture per day (e.g., by perfusion exchange). In some cases, the process comprises adding from about 0.2 g/L cys equivalents to less than 0.9 g/L cys equivalents to the culture per day (e.g., by perfusion exchange). In some cases, the process comprises adding from about 0.2 g/L cys equivalents to less than 0.6 g/L cys equivalents to the culture per day (e.g., by perfusion exchange). In some cases, the process comprises adding from about 0.2 g/L cys equivalents to less than 0.5 g/L cys equivalents to the culture per day (e.g., by perfusion exchange). In some cases, the process comprises adding from about 0.2 g/L cys equivalents to less than 0.4 g/L cys equivalents to the culture per day (e.g., by perfusion exchange).

In some embodiments, a secreted recombinant protein having a free cysteine is produced in a perfusion batch process by culturing mammalian cells in base perfusion media containing less than 0.62 g/L cys equivalents and continuously or discontinuously exchanging (e.g., by perfusion) all or a portion of the cell culture media with an exchange media containing less than 1.1 g/L cys equivalents. In some cases, the perfusion exchange media contains less than 1 g/L, less than 0.9 g/L, less than 0.6 g/L, less than 0.5 g/L, less than about 0.4 g/L or about 0.3 g/L cys equivalents. In some embodiments, the base perfusion media contains less than 0.6 g/L, or about 0.3 g/L cys equivalents.

In some embodiments, the base perfusion media contains from about 0.2 g/L cysteine to less than about 0.6 g/L cys equivalents. In some embodiments, the base perfusion media contains from about 0.25 g/L cysteine to less than about 0.5 g/L cys equivalents. In some embodiments, the base perfusion media contains from about 0.3 g/L cysteine to less than about 0.4 g/L cys equivalents. In some embodiments, the perfusion exchange media contains from about 0.2 g/L cysteine to less than about 1.1 g/L cys equivalents. In some embodiments, the perfusion exchange media contains from about 0.25 g/L cysteine to less than about 0.9 g/L cys equivalents. In some embodiments, the perfusion exchange media contains from about 0.3 g/L cysteine to less than about 0.6 g/L cys equivalents. In some embodiments, the perfusion base media contains no, or substantially no, cysteine. In some embodiments, the perfusion exchange media contains no, or substantially no, cysteine. In some embodiments, the perfusion base media and/or the perfusion exchange media contains no, or substantially no, cysteine. In some embodiments, the perfusion base media and the perfusion exchange media are identical or substantially identical. A production (e.g., a perfusion base or exchange production or fed batch base or feed) medium containing substantially no cysteine includes a media in which a residual amount of cysteine is present due to a presence of residual expansion culture media, cysteine production by the host cell, and/or reduction of cystine during the production culture. A base, exchange, or feed media containing substantially no cysteine contains less than 0.1 g/L of cysteine, preferably less than 0.05 g/L cysteine, more preferably less than 0.01 g/L cysteine.

In some embodiments, activity is measured by a cystamine-CEX (cation exchange chromatography) method. The cystamine-CEX method includes derivatization of the antibody with cystamine (2,2′-dithiobis(ethylamine)), followed by analytical separation using cation exchange chromatography (CEX). Because the activity of the antibodies disclosed herein (e.g., secukinumab) is decreased if CysL97 is in oxidized form, derivatization of CysL97 with cystamine serves as a proxy to measure antibody activity. Derivatization by cystamine leads to an addition of one positive charge per free Cys97 residue. The resulting derivatized forms of secukinumab (e.g., +2, +1 charges) can then be separated from the non-derivatized form and quantified by CEX. A cystamine-derivatized secukinumab molecule with two cystamine bound to unpaired Cys97 on both light chains may be considered 100% biological active in theory. A cystamine-derivatized secukinumab molecule with addition of one cystamine bound to unpaired Cys97 on one of the light chains may be considered 50% biological active. A cystamine-derivatized secukinumab molecule without any cystamine bound to the molecule may be considered biological inactive. The level of cystamine derivitization in a preparation of antibodies (e.g., a preparation of secukinumab antibodies), in comparison to the theoretical maximum level of cystamine derivitization in that preparation (e.g., expressed as a percentage of theoretical maximum) may then be used as a measure of the activity of the preparation.

In brief cystamine-CEX may be performed as follows. Antibody samples (50 μg) are first treated with carboxypeptidase B (1:40, w:w) to remove the C-terminal lysine in the heavy chain and then derivatized with 4 mM cystamine in 5 mM sodium acetate, 0.5 mM EDTA, pH4.7 at room temperature for 2 hours. The derivatization is stopped by addition of 2 μL of 1M phosphoric acid. CEX is performed on the cystamine-derivatized antibody samples using a ProPac™ WCX-10 analytical column (4 mm×250 mm, Dionex). A gradient from 12.5 mM to 92.5 mM sodium chloride in 25 mM sodium phosphate, pH 6.0 at a flow rate of 1.0 ml/min is used for separation. Absorption at 220 nm is recorded by a UV detector (Agilent HPLC 1200).

### IL-17 Antibodies and Antigen Binding Fragments Thereof

In one embodiment, the IL-17 antibody or antigen binding fragment thereof comprises at least one immunoglobulin heavy chain variable domain (V_{H}) comprising hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3. In one embodiment, the IL-17 antibody or antigen binding fragment thereof comprises at least one immunoglobulin light chain variable domain (V_{L′}) comprising hypervariable regions CDR1′, CDR2′ and CDR3′, said CDR1′ having the amino acid sequence SEQ ID NO:4, said CDR2′ having the amino acid sequence SEQ ID NO:5 and said CDR3′ having the amino acid sequence SEQ ID NO:6. In one embodiment, the IL-17 antibody or antigen binding fragment thereof comprises at least one immunoglobulin heavy chain variable domain (V_{H}) comprising hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13.

In one embodiment, the IL-17 antibody or antigen binding fragment thereof comprises at least one immunoglobulin V_{H} domain and at least one immunoglobulin V_{L} domain, wherein: a) the V_{H} domain comprises (e.g., in sequence): i) hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; or ii) hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13; and b) the V_{L} domain comprises (e.g., in sequence) hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antibody or antigen binding fragment thereof comprises: a) an immunoglobulin heavy chain variable domain (V_{H}) comprising the amino acid sequence set forth as SEQ ID NO:8; b) an immunoglobulin light chain variable domain (V_{L}) comprising the amino acid sequence set forth as SEQ ID NO:10; c) an immunoglobulin V_{H} domain comprising the amino acid sequence set forth as SEQ ID NO:8 and an immunoglobulin V_{L} domain comprising the amino acid sequence set forth as SEQ ID NO:10; d) an immunoglobulin V_{H} domain comprising the hypervariable regions set forth as SEQ ID NO:1, SEQ ID NO:2, and SEQ ID NO:3; e) an immunoglobulin V_{L} domain comprising the hypervariable regions set forth as SEQ ID NO:4, SEQ ID NO:5 and SEQ ID NO:6; f) an immunoglobulin V_{H} domain comprising the hypervariable regions set forth as SEQ ID NO:11, SEQ ID NO:12 and SEQ ID NO:13; g) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or h) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

For ease of reference the amino acid sequences of the hypervariable regions of the secukinumab monoclonal antibody, based on the Kabat definition and as determined by the X-ray analysis and using the approach of Chothia and coworkers, is provided in Table 1, below.

TABLE 1
Amino acid sequences of the hypervariable
regions of the secukinumab antibody
Light-Chain

| | | |
|---|---|---|
| CDR1′ | Kabat | R-A-S-Q-S-V-S-S-S-Y-L-A (SEQ ID |
| | | NO: 4) |
| | Chothia | R-A-S-Q-S-V-S-S-S-Y-L-A (SEQ ID |
| | | NO: 4) |
| CDR2′ | Kabat | G-A-S-S-R-A-T (SEQ ID NO: 5) |
| | Chothia | G-A-S-S-R-A-T (SEQ ID NO: 5) |
| CDR2′ | Kabat | Q-Q-Y-G-S-S-P-C-T (SEQ ID NO: 6) |
| | Chothia | Q-Q-Y-G-S-S-P-C-T (SEQ ID NO: 6) |

Heavy-Chain

| | | |
|---|---|---|
| CDR1 | Kabat | N-Y-W-M-N (SEQ ID NO: 1) |
| CDR1-x | Chothia | G-F-T-F-S-N-Y-W-M-N (SEQ ID NO: |
| | | 11) |
| CDR2 | Kabat | A-I-N-Q-D-G-S-E-K-Y-Y-V-G-S-V-K-G |
| | | (SEQ ID NO: 2) |
| CDR2-x | Chothia | A-I-N-Q-D-G-S-E-K-Y-Y (SEQ ID NO: |
| | | 12) |
| CDR3 | Kabat | D-Y-Y-D-I-L-T-D-Y-Y-I-H-Y-W-Y-F-D- |
| | | L (SEQ ID NO: 3) |
| CDR3-x | Chothia | C-V-R-D-Y-Y-D-I-L-T-D-Y-Y-I-H-Y-W- |
| | | Y-F-D-L-W-G (SEQ ID NO: 13) |

In preferred embodiments, the constant region domains preferably also comprise suitable human constant region domains, for instance as described in “Sequences of Proteins of Immunological Interest”, Kabat E.A. et al, US Department of Health and Human Services, Public Health Service, National Institute of Health. DNA encoding the V_{L} of secukinumab is set forth in SEQ ID NO:9. DNA encoding the V_{H} of secukinumab is set forth in SEQ ID NO:7.

In some embodiments, the IL-17 antibody or antigen binding fragment thereof (e.g., secukinumab) comprises the three CDRs of SEQ ID NO:10. In other embodiments, the IL-17 antibody or antigen binding fragment thereof comprises the three CDRs of SEQ ID NO:8. In other embodiments, the IL-17 antibody or antigen binding fragment thereof comprises the three CDRs of SEQ ID NO:10 and the three CDRs of SEQ ID NO:8. CDRs of SEQ ID NO:8 and SEQ ID NO:10 may be found in Table 1. The free cysteine in the light chain (CysL97) may be seen in SEQ ID NO:6.

In some embodiments, IL-17 antibody or antigen binding fragment thereof comprises the light chain of SEQ ID NO:14. In other embodiments, the IL-17 antibody or antigen binding fragment thereof comprises the heavy chain of SEQ ID NO:15 (with or without the C-terminal lysine). In other embodiments, the IL-17 antibody or antigen binding fragment thereof comprises the light chain of SEQ ID NO:14 and the heavy chain of SEQ ID NO:15 (with or without the C-terminal lysine). In some embodiments, the IL-17 antibody or antigen binding fragment thereof comprises the three CDRs of SEQ ID NO:14. In other embodiments, IL-17 antibody or antigen binding fragment thereof comprises the three CDRs of SEQ ID NO:15. In other embodiments, the IL-17 antibody or antigen binding fragment thereof comprises the three CDRs of SEQ ID NO:14 and the three CDRs of SEQ ID NO:15. CDRs of SEQ ID NO:14 and SEQ ID NO:15 may be found in Table 1. A complete set of sequences is found in Table .

Hypervariable regions may be associated with any kind of framework regions, though preferably are of human origin. Suitable framework regions are described in Kabat E. A. et al, ibid. The preferred heavy chain framework is a human heavy chain framework, for instance that of the secukinumab antibody. It consists in sequence, e.g. of FR1 (amino acid 1 to 30 of SEQ ID NO:8), FR2 (amino acid 36 to 49 of SEQ ID NO:8), FR3 (amino acid 67 to 98 of SEQ ID NO:8) and FR4 (amino acid 117 to 127 of SEQ ID NO:8) regions. Taking into consideration the determined hypervariable regions of secukinumab by X-ray analysis, another preferred heavy chain framework consists in sequence of FR1-x (amino acid 1 to 25 of SEQ ID NO:8), FR2-x (amino acid 36 to 49 of SEQ ID NO:8), FR3-x (amino acid 61 to 95 of SEQ ID NO:8) and FR4 (amino acid 119 to 127 of SEQ ID NO:8) regions. In a similar manner, the light chain framework consists, in sequence, of FR1′ (amino acid 1 to 23 of SEQ ID NO:10), FR2′ (amino acid 36 to 50 of SEQ ID NO:10), FR3′ (amino acid 58 to 89 of SEQ ID NO:10) and FR4′ (amino acid 99 to 109 of SEQ ID NO:10) regions.

In one embodiment, the IL-17 antibody or antigen binding fragment thereof (e.g., secukinumab) is selected from a human IL-17 antibody that comprises at least: a) an immunoglobulin heavy chain or fragment thereof comprising a variable domain comprising, in sequence, the hypervariable regions CDR1, CDR2 and CDR3 and the constant part or fragment thereof of a human heavy chain; said CDR1 having the amino acid sequence SEQ ID NO:1, said CDR2 having the amino acid sequence SEQ ID NO:2, and said CDR3 having the amino acid sequence SEQ ID NO:3; and b) an immunoglobulin light chain or fragment thereof comprising a variable domain comprising, in sequence, the hypervariable regions CDR1′, CDR2′, and CDR3′ and the constant part or fragment thereof of a human light chain, said CDR1′ having the amino acid sequence SEQ ID NO: 4, said CDR2′ having the amino acid sequence SEQ ID NO:5, and said CDR3′ having the amino acid sequence SEQ ID NO:6.

In one embodiment, the IL-17 antibody or antigen binding fragment thereof is selected from a single chain antibody or antigen binding fragment thereof that comprises an antigen binding site comprising: a) a first domain comprising, in sequence, the hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; and b) a second domain comprising, in sequence, the hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6; and c) a peptide linker which is bound either to the N-terminal extremity of the first domain and to the C-terminal extremity of the second domain or to the C-terminal extremity of the first domain and to the N-terminal extremity of the second domain.

Alternatively, an IL-17 antibody or antigen binding fragment thereof as used in the disclosed methods may comprise a derivative of the IL-17 antibodies set forth herein by sequence (e.g., a pegylated version of secukinumab). Alternatively, the V_{H} or V_{L} domain of an IL-17 antibody or antigen binding fragment thereof used in the disclosed methods may have V_{H} or VL domains that are substantially identical to the V_{H} or V_{L} domains set forth herein (e.g., those set forth in SEQ ID NO:8 and 10). A human IL-17 antibody disclosed herein may comprise a heavy chain that is substantially identical to that set forth as SEQ ID NO:15 (with or without the C-terminal lysine) and/or a light chain that is substantially identical to that set forth as SEQ ID NO:14. A human IL-17 antibody disclosed herein may comprise a heavy chain that comprises SEQ ID NO:15 (with or without the C-terminal lysine) and a light chain that comprises SEQ ID NO:14. A human IL-17 antibody disclosed herein may comprise: a) one heavy chain which comprises a variable domain having an amino acid sequence substantially identical to that shown in SEQ ID NO:8 and the constant part of a human heavy chain; and b) one light chain which comprises a variable domain having an amino acid sequence substantially identical to that shown in SEQ ID NO:10 and the constant part of a human light chain.

Alternatively, an IL-17 antibody or antigen binding fragment thereof used in the disclosed methods may be an amino acid sequence variant of the reference IL-17 antibodies set forth herein, as long as it contains CysL97. The disclosure also includes IL-17 antibodies or antigen binding fragments thereof (e.g., secukinumab) in which one or more of the amino acid residues of the VH or V_{L} domain of secukinumab (but not CysL97), typically only a few (e.g., 1-10), are changed; for instance by mutation, e.g., site directed mutagenesis of the corresponding DNA sequences. In all such cases of derivative and variants, the IL-17 antibody or antigen binding fragment thereof is capable of inhibiting the activity of about 1 nM (=30 ng/ml) human IL-17 at a concentration of about 50 nM or less, about 20 nM or less, about 10 nM or less, about 5 nM or less, about 2 nM or less, or more preferably of about 1 nM or less of said molecule by 50%, said inhibitory activity being measured on IL-6 production induced by hu-IL-17 in human dermal fibroblasts as described in Example 1 of WO 2006/013107.

In some embodiments, the IL-17 antibodies or antigen binding fragments thereof, e.g., secukinumab, bind to an epitope of mature human IL-17 comprising Leu74, Tyr85, His86, Met87, Asn88, Va1124, Thr125, Pro126, Ile127, Va1128, His129. In some embodiments, the IL-17 antibody, e.g., secukinumab, binds to an epitope of mature human IL-17 comprising Tyr43, Tyr44, Arg46, Ala79, Asp80. In some embodiments, the IL-17 antibody, e.g., secukinumab, binds to an epitope of an IL-17 homodimer having two mature human IL-17 chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain. The residue numbering scheme used to define these epitopes is based on residue one being the first amino acid of the mature protein (ie., IL-17A lacking the 23 amino acid N-terminal signal peptide and beginning with Glycine). The sequence for immature IL-17A is set forth in the Swiss-Prot entry Q16552. In some embodiments, the IL-17 antibody has a K_{D} of about 100-200 pM. In some embodiments, the IL-17 antibody has an IC₅₀ of about 0.4 nM for in vitro neutralization of the biological activity of about 0.67 nM human IL-17A. In some embodiments, the absolute bioavailability of subcutaneously (s.c.) administered IL-17 antibody has a range of about 60-about 80%, e.g., about 76%. In some embodiments, the IL-17 antibody, such as secukinumab, has an elimination half-life of about 4 weeks (e.g., about 23 to about 35 days, about 23 to about 30 days, e.g., about 30 days). In some embodiments, the IL-17 antibody (such as secukinumab) has a Tₘₐₓ of about 7-8 days.

Particularly preferred IL-17 antibodies or antigen binding fragments thereof used in the disclosed methods are human antibodies, especially secukinumab as described in Examples 1 and 2 of WO 2006/013107. Secukinumab is a recombinant high-affinity, fully human monoclonal anti-human interleukin-17A (IL-17A, IL-17) antibody of the IgG1/kappa isotype that is currently in clinical trials for the treatment of immune-mediated inflammatory conditions. Secukinumab (see, e.g., WO2006/013107 and WO2007/117749) has a very high affinity for IL-17, i.e., a K_{D} of about 100-200 pM and an IC₅₀ for in vitro neutralization of the biological activity of about 0.67 nM human IL-17A of about 0.4 nM. Thus, secukinumab inhibits antigen at a molar ratio of about 1:1. This high binding affinity makes the secukinumab antibody particularly suitable for therapeutic applications. Furthermore, it has been determined that secukinumab has a very long half-life, i.e., about 4 weeks, which allows for prolonged periods between administration, an exceptional property when treating chronic life-long disorders, such as rheumatoid arthritis.

Disclosed herein are processes for preparation of the above-mentioned IL-17 antibodies and antigen binding fragments thereof (e.g., secukinumab). The disclosed methods conveniently may be performed on preparations of antibodies (e.g., IL-17 antibodies, e.g., secukinumab) to reduce cost. A “preparation” of antibodies refers to a composition (e.g., solution) having a plurality of an antibody molecule. A “preparation” includes any liquid composition comprising the IL-17 antibody or antigen binding fragment thereof. As such, a preparation may comprise, e.g., IL-17 antibody or antigen binding fragment thereof, e.g., secukinumab, in water or a buffer, in a column elutate, in a dialysis buffer, etc. In some embodiments, the initial preparation of antibodies comprises a pool of the IL-17 antibodies or antigen binding fragments thereof, e.g., secukinumab, in a buffer (e.g., a Tris, e.g., 1 mM-1M Tris, pH 6.0-8.0) or WFI.

In some embodiments of the above methods, the IL-17 antibody or antigen binding fragment thereof comprises: i) an immunoglobulin heavy chain variable domain (VH) comprising the amino acid sequence set forth as SEQ ID NO:8; ii) an immunoglobulin light chain variable domain (VL) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10; iv) an immunoglobulin V_{H} domain comprising, in sequence, the hypervariable regions set forth as SEQ ID NO:1, SEQ ID NO:2, and SEQ ID NO:3; v) an immunoglobulin V_{L} domain comprising, in sequence, the hypervariable regions set forth as SEQ ID NO:4, SEQ ID NO:5 and SEQ ID NO:6; vi) an immunoglobulin V_{H} domain comprising, in sequence, the hypervariable regions set forth as SEQ ID NO:11, SEQ ID NO:12 and SEQ ID NO:13; vii) an immunoglobulin VH domain comprising, in sequence, the hypervariable regions set forth as SEQ ID NO:1, SEQ ID NO:2, and SEQ ID NO:3 and an immunoglobulin V_{L} domain comprising, in sequence, the hypervariable regions set forth as SEQ ID NO:4, SEQ ID NO:5 and SEQ ID NO:6; and viii) an immunoglobulin V_{H} domain comprising, in sequence, the hypervariable regions set forth as SEQ ID NO:11, SEQ ID NO:12 and SEQ ID NO:13 and an immunoglobulin V_{L} domain comprising, in sequence, the hypervariable regions set forth as SEQ ID NO:4, SEQ ID NO:5 and SEQ ID NO:6. In some embodiments of the disclosed methods, the IL-17 antibody or antigen binding fragment thereof is a human antibody of the IgGi isotype. In some embodiments of the disclosed methods, the antibody is secukinumab.

### Recombinant Antibody Production

The manufacturing of recombinant polypeptides is traditionally divided in two main steps: upstream (cell culture and synthesis of the target polypeptide) and downstream (purification and formulation of the polypeptide into a drug substance or drug product).

More specifically, preparations of monoclonal antibodies or antigen binding fragments thereof may be recombinantly produced by any mammalian cells using any mammalian cell line, e.g., Chinese hamster ovary cells (CHO) cells, mouse myeloma NSO cells, baby hamster kidney (BHK) cells, human embryonic kidney cell line HEK-293, the human retinal cell line Per.C6 (Crucell, NL), HKB11 cell clone (derived from a hybrid cell fusion of HEK 293S with the Burkitt's lymphoma line 2B8), etc. By “recombinantly produced by mammalian cells” is meant that production of the antibody in the mammalian cells has been achieved using recombinant DNA technology.

CHO cells are currently the most widely used mammalian hosts in biological and medical research and particularly for the expression of human therapeutic proteins and it has been reported that around 70% of recombinant therapeutic proteins are produced in CHO cell systems. Although they require expensive culture media and grow relatively slowly in comparison with E. colli and yeast expression systems, CHO cells enable more accurate protein glycosylation, assembly and folding like human cells. Therefore, for some proteins whose activities are closely linked to posttranslational modification CHO cells are the preferred production hosts. CHO cells also synthesize efficiently extremely large molecules that are unable to be expressed actively in prokaryote hosts. Suitable CHO cell lines include e.g. CHO-S (Invitrogen, Carlsbad, Calif., USA), CHO Kl (ATCC CCL-61), CHO pro3-, CHO DG44, CHO P12 or the dhfr- CHO cell line DUK-BII (Urlaub G & Chasin L A (1980) PNAS 77(7): 4216-4220), DUXBI 1 (Simonsen CC & Levinson A D (1983) PNAS 80(9): 2495-2499), or CHO-K1SV (Lonza, Basel, Switzerland). Many CHO cell-derived products have received regulatory approval such as erythropoietin (Epogen; Amgen), TNFα receptor fusion (Enbrel; Amgen), anti-HER2 antibody (Herceptin; Genentech), anti-TNFα antibody (Humira; Abbvie) and anti-VEGF antibody (Avastin; Genentech).

For the industrial production of recombinant proteins, the most common cultivation modes used in biomanufacturing are fed-batch and perfusion. The use of one or the other technology depends on different factors linked to the protein or the host (Kadouri & Spier, (1997) Cytotechnology 24: 89-98), with cells cultivated attached on carriers or in suspension. One of the most common processes is the batch bioreactor where after inoculation, cells grow and produce until a limitation due to media consumption is reached and cell density starts to decrease. The second very common process is fed-batch where nutrient limitations are prevented by adding highly concentrated feeds at different time points during the cultivation. The culture duration is therefore longer than in batch mode and the final productivity is increased. For continuous processes where media is fed continuously and harvest is removed continuously, one of the simplest is a chemostat process where media is added at a constant flowrate and the bioreactor content is removed at the same flowrate, without any cell retention (Henry O et al., (2008) Biotechnol. Prog., 921-931). An alternative continuous process is perfusion where there is a constant in and out flow but the cells are now retained inside the bioreactor. The current industry standard for the production of stable proteins such as monoclonal antibodies is a fed-batch process in stirred tank bioreactors of up to 20 kL. These cultivation vessels can provide very high mixing and mass transfer rates and also provide high flexibility for the working volume and can be used for different cell types and operation modes (Rodrigues M E et al., (2010) Biorechnol. Prog. 26: 332-51).

Media development has been the most important aspect of cell culture development and optimization since the beginning of biomanufacturing, firstly for process performance but secondly, also and more importantly, for safety reasons. The first cell culture media were prepared using animal derived products (Yao & Asayama, (2017) Reprod. Med. Biol., 16: 99-117). The consequence for the patients was the exposure to many hazardous factors such as viruses and prions, and infection risks for patients were important, especially for chronic diseases because the patients were continuously exposed to the drug (Grillberger L et al., (2009) Biotechnol. J., 4: 186-201). Process inconsistency due to batch heterogeneity was also a driver to reduce animal or even plant derived media components and even today significant efforts are spent in the optimization of chemically defined media that can enhance cell growth.

Chemically defined media are now available commercially and most of the large biomanufacturing companies have developed their own formulations. Highly concentrated feed, for example, can be challenging because of the physical properties of some compounds that cause solubility or stability constraints. Further optimization of CHO cell culture media and process parameters, specifically aimed at commercial manufacturing of monoclonal antibodies and other recombinant polypeptides, has resulted in dramatic increases in cell density and protein expression, with titers reaching more than 10 g/L in some cases (Li F et al., (2010) MAbs, 2(5): 466-479; Lu F et al., (2013) Biotechnol Bioeng., 110(1): 191-205; Xing Z et al., (2011) Process Biochem., 46(7): 1432-9). Several companies specializing in cell culture media have developed and optimized basal and feed media combinations specifically for recombinant CHO manufacturing processes (ThermoFisher, Waltham, Mass., USA, GE Healthcare, Waukesha, Wis., USA, MilliporeSigma, St. Louis, Mo., USA, Lonza, Basel, Switzerland, Irvine Scientific, Santa Ana, Calif., USA).

The formulations of these commercially available media are typically proprietary; however all cell culture media require similar basic nutrients, which are essential to support life and cellular growth. Water, along with sources of carbon, nitrogen and phosphate, certain amino acids, fatty acids, vitamins, trace elements and salts are all supplied in concentrations based on the chemical makeup of the cell, the calculated amounts required to reach a desired cell density and knowledge of nutrient depletion rates so that critical components may be replenished to maintain and extend cell viability. In particular, amino acids are key components in CHO cell culture media, especially in chemically defined media and studies have shown that small changes in amino acid composition of cell culture media can alter growth profiles and titers, and can also significantly affect product glycosylation patterns (Fan Y et al., (2015) Biotechnol. Bioeng., 112(3): 521-35).

In general, amino acids may be classified into nonessential amino acids, which can be synthesized by mammalian cells, and essential amino acids, which cells are unable to synthesize and must therefore be supplied as components of the cell culture medium. Both nonessential amino acids and essential amino acids can have significant effects on CHO cell growth and optimization of the relative concentrations of nonessential amino acids and essential amino acids in the media formulation has been shown to improve the productivity of a recombinant monoclonal antibody (Parampalli A et al., (2007) Cytotechnology, 54(1): 57-68). Essential amino acids include histidine, isoleucine, leucine, lysine, methionine, phenylalanine, threonine, tryptophan, and valine, and in most instances, all of the essential amino acids are required in CHO cell culture media.

The nonessential amino acids include alanine, arginine, asparagine, aspartic acid, cysteine, glutamic acid, glutamine, glycine, proline, serine, and tyrosine. Despite the fact that the nonessential amino acids can be synthesized by mammalian cells in culture, most cell culture media still contain most or all of these amino acids to support cell growth and polypeptide production. Most of the nonessential amino acids can have a significant effect on cell culture processes.

In particular, cysteine, the only thiol-containing amino acid, is a special nonessential amino acid in monoclonal antibody production. The formation of disulfide bridges between sulfhydryl groups on cysteine residues supports the folding of tertiary and quaternary structure of both CHO cell structural proteins and recombinant antibody product. Cysteine limitation can be fatal and irreversible for CHO cell growth, and may lead to a cell viability drop. In a study by Ghaffari et al (2020), the effect of limiting glutamine, asparagine, and cysteine on the cell growth, metabolism, antibody productivity and product glycosylation was investigated in three Chinese hamster ovary (CHO) cell lines (CHO-DXB11, CHO-K1SV, and CHO-S). Cysteine limitation was detrimental to both cell proliferation and productivity for all three CHO cell lines. Of the three amino acid limitations studied, the cysteine limitation had the greatest detrimental impact on the culture growth and productivity, as well as the mAb glycosylation. Cysteine has a low solubility and can become limiting in the discontinuous feeding protocols commonly used at industrial scales, especially at high cell concentrations. Ghaffari et al investigated the duration that the CHO-DXB11 cells could tolerate cysteine limitation, with these cells initially grown in BIOGRO medium with no cysteine. The cysteine concentration was then restored to 0.4 mM on either Day 1 or Day 2 of the culture by the addition of a concentrated cysteine solution. If the cysteine level was restored on Day 1, the cells remained in a lag phase for an extra day and then resumed growth on Day 2; by Day 5 these cultures reaching concentrations similar to the control. Restoring the cysteine levels after 2 days of the cysteine limited culture proved to be ineffective and the cells did not grow. (Ghaffari N et al., (2020) Biotech. Prog., 36: e2946). In contract, cysteine concentration of >1 mM can be toxic for mammalian cells, possibly due to lipid peroxidation and formation of hydroxyl radicals, which can be further accelerated in the presence of copper (Ritacco F V et al (2018) Biotechnol. Prog., 34(6): 1407-26). In mammals, the cysteine pool is regulated by the liver and without such a regulating mechanism in CHO cells, cysteine concentration in the medium needs to be carefully designed and controlled for cell culture processes (Stipanuk M H et al., (2006) J. Nutr., 136(6): 1652S-59S).

The recombinant polypeptide preparations, e.g., of IL-17 antibodies or antigen binding fragments thereof, for use in the processes described herein may be recombinantly produced by any mammalian cells using any mammalian cell line. Preferably the mammalian cell line is CHO cells. The recombinant polypeptides, e.g., anti-IL-17 antibodies or antigen binding fragments thereof, maybe produced in a continuous manufacturing system or using a fed-batch system with the addition of feeds to the culture media. As described above, the anti-IL-17 antibody secukinumab comprises a free, unpaired cysteine that is involved in antigen recognition and binding. This free cysteine residue is found after the cis-proline in the light chain complementarity determining region (CDR) 3 loop i.e., amino acid eight of L-CDR3 as set forth as SEQ ID NO:6, which corresponds to amino acid 97 of the light chain variable region as set forth as SEQ ID NO:10, and is referred to as “CysL97”. In order to maintain full activity, this free cysteine residue cannot be masked by oxidative disulfide pairing with other cysteine residues or by oxidation with exogenous compounds. Furthermore, modification of this free cysteine can have a negative effect on the activity and stability of the antibody and can lead to increased immunogenicity. Therefore, processing of secukinumab can be difficult, as the end product may contain a substantial amount of inactive antibody material. However, because secukinuamb is manufactured using mammalian cells, in particular CHO cells, cell-based modifications of CysL97 do occur, which can impact yield and antibody activity.

As discussed above, the term “cys-equivalents” refers to the cysteine and cystine available to the cells in the culture medium, in the culture vessel whether derived from the base medium and/or feed medium. Surprisingly, it has been found that lowering the amount of cys equivalents in the cell culture growth media of secukinumab results in a decrease in the modification of the free cysteine CysL97. This in turn results in an increase in active antibody that is produced by the production process, i.e. an increase in product quality. Contrary to established studies (e.g. Ghaffari et al, supra), a decrease in cysteine in the production media and media feeds, had no negative effect on the yield of secukinumab.

### Purification of Recombinant Polypeptide

To obtain substantially homogeneous preparations of the recombinant polypeptides that are produced according to the cell culture processes as described herein, a purification step is necessary. As a first step, the culture medium or lysate is normally centrifuged to remove particulate cell debris. The produced polypeptides can be conveniently purified by hydroxyapatite chromatography, gel electrophoresis, dialysis, or affinity chromatography. Other techniques for protein purification such as fractionation on an ion-exchange column, ethanol precipitation, reverse phase HPLC, chromatography on silica, chromatography on heparin Sepharose, chromatography on an anion or cation exchange resin (such as a polyaspartic acid column), chromatofocusing, SDS-PAGE, and ammonium sulfate precipitation are also available.

### Pharmaceutical Compositions, Administration and Kits

Provided herein are pharmaceutical compositions comprising a recombinant polypeptide as described herein, in combination with one or more pharmaceutically acceptable excipient, diluent or carrier. To prepare pharmaceutical or sterile compositions including a molecule of the present disclosure, the molecule is mixed with a pharmaceutically acceptable carrier or excipient. The phrase “pharmaceutically acceptable” means approved by a regulatory agency of a federal or a state government, or listed in the U.S. Pharmacopeia or other generally recognized pharmacopeia for use in animals, and more particularly, in humans. The term “pharmaceutical composition” refers to a mixture of at least one active ingredient (e.g., an antibody or fragment of the disclosure) and at least one pharmaceutically-acceptable excipient, diluent or carrier. A “medicament” refers to a substance used for medical treatment.

Pharmaceutical compositions of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al., (eds.) (1993) Pharmaceutical Dosage Forms: parenteral Medications, Marcel Dekker, NY; Lieberman, et al., (eds.) (1990) Pharmaceutical Dosage Forms: Tablets, Marcel Dekker, NY; Lieberman, et al., (eds.) (1990) Pharmaceutical Dosage Forms: Disperse Systems, Marcel Dekker, NY; Weiner & Kotkoskie (2000) Excipient Toxicity and Safety, Marcel Dekker, Inc., New York, N.Y.).

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al., (2003) New Engl. J. Med. 348:601-608; Milgrom et al., (1999) New Engl. J. Med. 341:1966-1973; Slamon, et al. (2001) New Engl. J. Med. 344:783-792; Beniaminovitz et al., (2000) New Engl. J. Med. 342:613-619; Ghosh et al., (2003) New Engl. J. Med. 348:24-32; Lipsky et al., (2000) New Engl. J. Med. 343:1594-1602).

The present disclosure further encompasses kits for treating a patient having a pathological disorder mediated by IL-17, e.g., an autoimmune disease or an inflammatory disorder or condition. Such kits comprise a therapeutically effective amount of an antibody produced according to a process described herein and a package leaflet, in which the package leaflet indicates the recommended dose regimen of the anti-IL-17 antibody for the patient. Preferably the antibody is an anti-IL-17 antibody such as secukinumab. Additionally, such kits may comprise means for administering the antibody (e.g., an autoinjector, a syringe and vial, a prefilled syringe, a prefilled pen) and instructions for use. Kits may also comprise instructions for administration of the anti-IL-17 antibody to treat the patient. Such instructions may provide the dose, route of administration, regimen, and total treatment duration for use with the enclosed antibody. The phrase “means for administering” is used to indicate any available implement for systemically administering a drug to a patient, including, but not limited to, a pre-filled syringe, a vial and syringe, an injection pen, an auto-injector, an IV drip and bag, an infusion pump, a patch, an infusion bag and needle, etc. With such items, a patient may self-administer the drug (i.e., administer the drug without the assistance of a physician) or a medical practitioner may administer the drug.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed patient matter, as defined by the appended claims.

### EXAMPLES

The following experiments are intended to further illustrate the invention as defined in this application.

### Example 1

A set of experiments was designed to see if cys97 oxidation of secukinumab occurs extracellularly.

Purified secukinumab drug substance was diafiltered (Amicon Ultracel tubes) with cell culture media that has standard amount of cysteine/cystine. Then, the solution was diluted in the media to a concentration of 1.5 g/L to be aligned with bioreactor titer. After this, the solution was incubated at 37° C. Finally, samples were taken after 24 and 48 hours for cystamine-CEX for free cys97 percentage.

Table 2 shows that free cys97 percentage decreases significantly after media incubation which suggests that cys97 can be oxidized in extracellular environment containing cysteine/cystine after secukinumab is secreted. This understanding led to the hypothesis that cys97 oxidation could occur due to the presence of cysteine/cystine in the cell culture medium.

TABLE 2
Secukinumab drug substance incubation with cell culture media

| | | Incubation | |
|---|---|---|---|
| Sample Name | Time | Temp | Free cys97% |
| Sample 1 | Day 0 | | 98.4 |
| Sample 2 | Day 1 | 37° C. | 57.4 |
| Sample 3 | Day 2 | 37° C. | 25.2 |

The results confirmed that cysteine/cystine in the cell culture media oxidizes cys97.

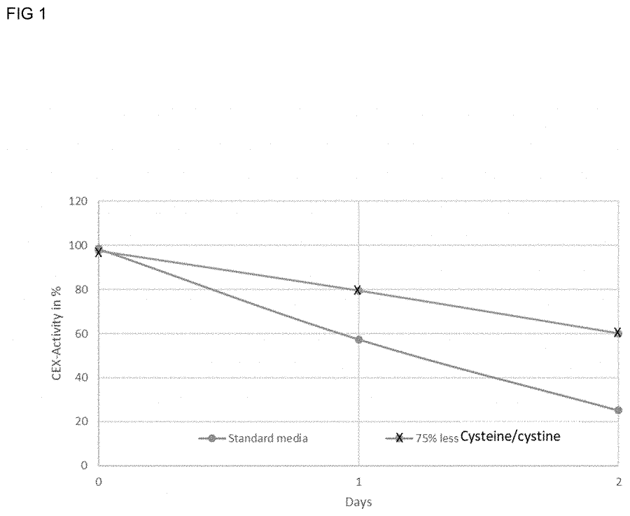

Secukinumab drug substance was incubated with media that contains different amount of cysteine/cystine. FIG. 1 shows that a lower amount of cysteine/cystine in the media reduces the amount the oxidation of cys97. This media incubation study suggests that cysteine/cystine does oxidize cys97 on secukinumab.

### Example 2

Previous observations had shown that incubation of secukinumab in media such as a perfusion medium at 37° C. resulted in a decrease in antibody activity over time. To determine whether the levels of cysteine/cystine, i.e. cys equivalents, in the media could diminish the drop of activity, secukinumab eluate was incubated in different variations of media based on a standard perfusion media to investigate the effect of media components.

To minimize dilution effects when adding sample to the media, the starting solution was diafiltrated in perfusion media. The obtained solution was added to perfusion media to achieve a final volume of 50 ml and a final protein concentration of ca. 1.5 g/L to mimic a typical antibody bioreactor titer. The solutions were incubated at 37° C. (standard bioreactor temperature) for two days. After 24 and 48 h, 25 ml samples were taken and the antibody captured. All samples were analyzed by Cystamine-CEX for activity. In the standard perfusion media, cysteine was supplied in the form of cysteine hydrochloride monohydrate and cystine was supplied from a stock solution comprising tyrosine and cystine.

Secukinumab was incubated in three media variants to investigate the influence of the media composition:

TABLE 3

| Media variant | Media change |
|---|---|
| Perfusion media | baseline, standard media |
| Variant 1 | baseline media |
| 75% reduction of total | without Cysteine HCl•H₂O |
| cysteine/cystine | with 50% reduction in Tyrosine/Cystine |
| concentration | stock solution |
| Variant 2 | baseline media |
| removal of cysteine/cystine | without Cysteine HCl•H₂O |
| | without Tyrosine/Cystine stock solution |
| | NaCl concentration adjusted to match same |
| | medium osmolality |
| Variant 3 | baseline media |
| removal of cysteine/cystine, | without Cysteine HCl H₂O |
| removal of trace elements | without Tyrosine/Cystine stock solution |
| | without trace elements, glycine |
| | NaCl concentration adjusted to match same |
| | medium osmolality |

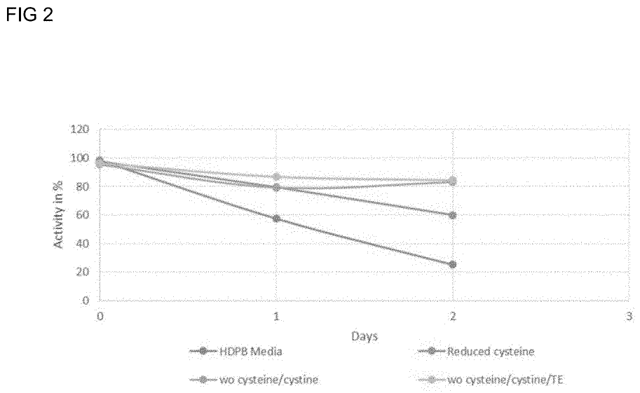

As shown in FIG. 2, activity of secukinumab dropped significantly when incubated in baseline media from around 98% down to 25% in 2 days. By reducing the amount of cys equivalents by 75%, the activity drop was reduced, dropping to 60%. Without any cys equivalents, the activity drop was further reduced, dropping to only 83%. Additionally removing trace elements did not show a significant improvement as activity dropped from 96% to 84%.

### Example 3

To determine the effect of the change in cysteine/cystine, i.e. cys equivalents, in a fed batch reactor method, an experiment was designed based on standard principles of CHO cell expression of secukinumab. The concentration of cysteine was varied in both the base media and in the feed media. No change was made to the concentration of cystine added from a tyrosine/cystine stock solution. The media variations tested are given in Table 4 below with the fed batch cell culture run for 10 days. Even with the reduction or removal of cysteine from the base or feed medium, cystine was still present in the media from the tyrosine/cystine stock solution. Therefore, the total cys equivalents for the baseline and media variants are also given in Table 4.

TABLE 4

| | | Cys | Cumulative cys |
|---|---|---|---|
| | | equivalents | equivalents added |
| | | in base and | to the cell |
| | | feed media | culture at |
| Media | Media description | (g/L) | Day 10 (g/L) |
| Baseline | Standard base medium (1:1 | 0.62 | 0.61 |
| | cysteine to cystine) | | |
| | Standard feed medium | 1.10 | |
| Variant 1 | no cysteine in base medium | 0.31 | 0.37 |
| | 40% reduction of cysteine in | 0.76 | |
| | feed medium | | |
| Variant 2 | no cysteine in base medium | 0.31 | 0.32 |
| | 70% reduction of cysteine in | 0.33 | |
| | feed medium | | |
| Variant 3 | no cysteine in base medium | 0.31 | 0.26 |
| | no cysteine in feed medium | 0.31 | |

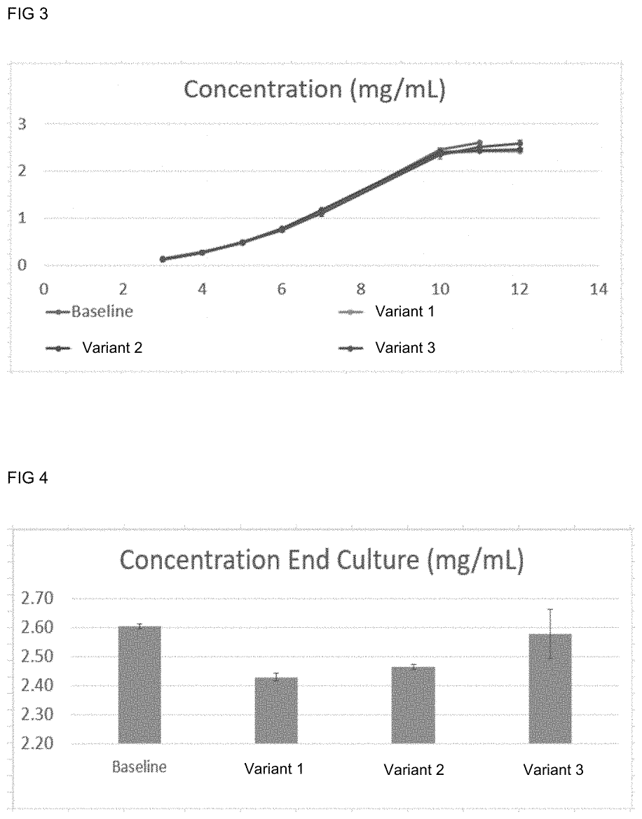

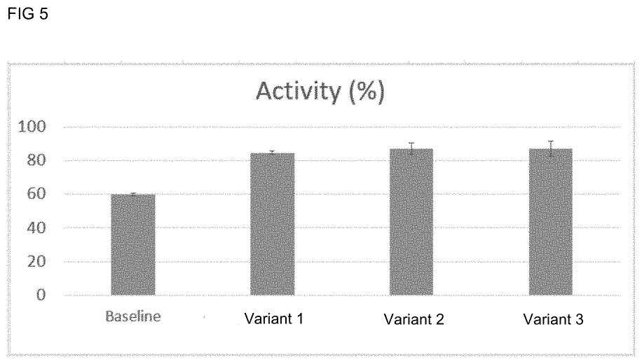

As shown in FIG. 3, varying the content of cys equivalents in the base media and media feed resulted in cell growth and expression titers of secukinumab (mg/ml) that were similar for all variants, including baseline. Only minor changes in the final antibody titer (mg/ml), were detected with the media variants (approximately 2.4 mg/ml with Variant 1 to approximately 2.6 mg/ml with Variant 3), as shown in FIG. 4.

FIG. 5 is a graph showing the activity (%) of secukinumab expressed using the different media variants. The activity of secukinumab expressed in cell culture medium with reduced cys equivalents was above 80%. In contrast, the activity of secukinumab expressed in the baseline cell culture medium was around 60%.

As demonstrated by this experiment, reduction and/or removal of cys equivalents from the base medium and/or feed media had no effect on yield of secukinumab in a fed batch process and furthermore, resulted in antibody product having a higher activity. Thus, these results suggest that secukinumab expressed with reduced concentrations of cys equivalents in a cell culture medium mitigates undesired cell-based modifications of CysL97, improving product quality.

### Example 4

An integrated drug substance manufacturing process employing a high cell density perfused batch (HDPB) culture at 1000 L scale, with approximately one reactor volume of perfused medium per day is adapted for production of secukinumab from CHO cells. The peak viable cell density (VCD) of the HDPB production process is nearly 16 million cells/mL and the process duration is approximately 19 days. Compared with fed-batch production medium, the HDPB production medium is minimally adjusted (concentration of components including manganese, pluronic F68, glucose, glutamine, cysteine, NaCl) to ensure growth robustness and desired product quality. No new components were introduced. The HDPB bioreactor volumetric productivity is approximately 1.2 g/L/day (or 22.8 g/L accumulated titer).

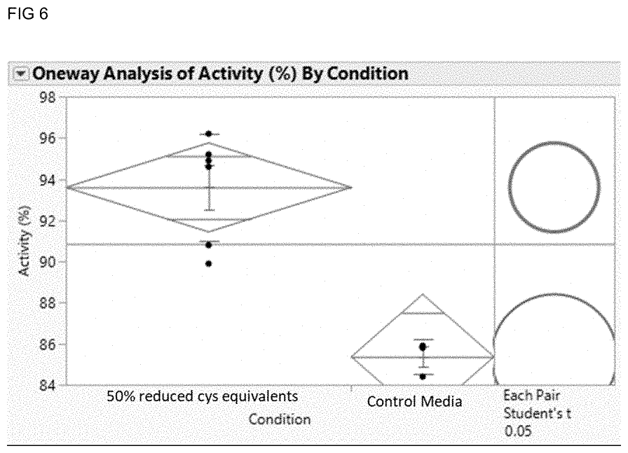

During evaluation of candidate media formulations, it was identified that reducing the cysteine concentration increased the bioactivity of secukinumab as measured by cystamine CEX. As shown in Tables 5 and 6 and FIG. 6, bioactivity was increased by approximately 6-8% at the lab scale experimental harvest pool, by reducing the cysteine concentration in the perfusion media by 50%. In addition to the identified effect in the upstream process, this was also evaluated from a downstream perspective due to the direct implications on the reduction step, which confirms the presented cysteine effect.

TABLE 5
Perfusion Media

| | | Cys equivalents | Cumulative cys |
|---|---|---|---|
| | | in perfusion | equivalents added |
| | | medium | to the cell culture at |
| Media | Media description | (g/L) | Day 19 (g/L) |
| Baseline | Standard perfusion | 0.615 | 11.642 |
| | medium (1:1 | | |
| | cysteine to cystine) | | |
| Variant 1 | no cysteine in | 0.305 | 5.785 |
| | perfusion medium | | |

TABLE 6
Perfusion results

| | | | Ac- | | Stand- |
|---|---|---|---|---|---|
| | | | tiv- | Aver- | ard |
| | | Analytics Lab | ity | age | Devia- |
| Condition | Batch No. | Sample Name | (%) | (%) | tion |
| Normal | 17494A-Q17 | 071, 101, 141, 171-HT723 | 85.9 | 85.7 | 0.3 |
| cys. | 17579A-Q10 | 071, 101, 151-HT742 | 84.3 | | |
| equivalents | 17582A-Q13 | 071, 101, 151, 171-HT742 | 85.8 | | |
| −50% cys | 17398A-S09 | HT697 | 96.2 | 93.6 | 2.6 |
| equivalents | 17399A-S10 | HT697 | 95.2 | | |
| | 17498A-Q21 | 071, 101, 141, 171-HT724 | 90.8 | | |
| | 17500A-Q23 | 071, 101, 141, 171-HT724 | 94.9 | | |
| | 17591A-S09 | POOL-HT746 | 89.9 | | |
| | 17593A-S11 | POOL-HT746 | 94.6 | | |

The lower cysteine concentration resulted in lower acidic profile when compared to the other conditions, which was also considered beneficial.

TABLE 7
Sequence Table

| Sequence ID | Sequence |
|---|---|
| SEQ ID NO: 1 | NYWMN |
| SEQ ID NO: 2 | AINQDGSEKYYVGSVKG |
| SEQ ID NO: 3 | DYYDILTDYYIHYWYFDL |
| SEQ ID NO: 4 | RASQSVSSSYLA |
| SEQ ID NO: 5 | GASSRAT |
| SEQ ID NO: 6 | QQYGSSPCT |
| SEQ ID NO: 7 | GAGGTGCAGTTGGTGGAGTCTGGGGGAGGCTTGGTCCAGCCTGGG |
| | GGGTCCCTGAGACTCTCCTGTGCAGCCTCTGGATTCACCTTTAGT |
| | AACTATTGGATGAACTGGGTCCGCCAGGCTCCAGGGAAAGGGCTG |
| | GAGTGGGTGGCCGCCATAAACCAAGATGGAAGTGAGAAATACTAT |
| | GTGGGCTCTGTGAAGGGCCGATTCACCATCTCCAGAGACAACGCC |
| | AAGAACTCACTGTATCTGCAAATGAACAGCCTGAGAGTCGAGGAC |
| | ACGGCTGTGTATTACTGTGTGAGGGACTATTACGATATTTTGACC |
| | GATTATTACATCCACTATTGGTACTTCGATCTCTGGGGCCGTGGC |
| | ACCCTGGTCACTGTCTCCTCA |
| SEQ ID NO: 8 | EVQLVESGGGLVQPGGSLRLSCAASGFTFSNYWMNWVRQAPGKGL |
| | EWVAAINQDGSEKYYVGSVKGRFTISRDNAKNSLYLQMNSLRVED |
| | TAVYYCVRDYYDILTDYYIHYWYFDLWGRGTLVTVSS |
| SEQ ID NO: 9 | GAAATTGTGTTGACGCAGTCTCCAGGCACCCTGTCTTTGTCTCCA |
| | GGGGAAAGAGCCACCCTCTCCTGCAGGGCCAGTCAGAGTGTTAGC |
| | AGCAGCTACTTAGCCTGGTACCAGCAGAAACCTGGCCAGGCTCCC |
| | AGGCTCCTCATCTATGGTGCATCCAGCAGGGCCACTGGCATCCCA |
| | GACAGGTTCAGTGGCAGTGGGTCTGGGACAGACTTCACTCTCACC |
| | ATCAGCAGACTGGAGCCTGAAGATTTTGCAGTGTATTACTGTCAG |
| | CAGTATGGTAGCTCACCGTGCACCTTCGGCCAAGGGACACGACTG |
| | GAGATTAAACGA |
| SEQ ID NO: 10 | EIVLTQSPGTLSLSPGERATLSCRASQSVSSSYLAWYQQKPGQAP |
| | RLLIYGASSRATGIPDRFSGSGSGTDFTLTISRLEPEDFAVYYCQ |
| | QYGSSPCTFGQGTRLEIKR |
| SEQ ID NO: 11 | GFTFSNYWMN |
| SEQ ID NO: 12 | AINQDGSEKYY |
| SEQ ID NO: 13 | CVRDYYDILTDYYIHYWYFDLWG |
| SEQ ID NO: 14 | EIVLTQSPGTLSLSPGERATLSCRASQSVSSSYLAWYQQKPGQAP |
| | RLLIYGASSRATGIPDRFSGSGSGTDFTLTISRLEPEDFAVYYCQ |
| | QYGSSPCTFGQGTRLEIKRTVAAPSVFIFPPSDEQLKSGTASVVC |
| | LLNNFYPREAKVQWKVDNALQSGNSQESVTEQDSKDSTYSLSSTL |
| | TLSKADYEKHKVYACEVTHQGLSSPVTKSFNRGEC |
| SEQ ID NO: 15 | EVQLVESGGGLVQPGGSLRLSCAASGFTFSNYWMNWVRQAPGKGL |
| | EWVAAINQDGSEKYYVGSVKGRFTISRDNAKNSLYLQMNSLRVED |
| | TAVYYCVRDYYDILTDYYIHYWYFDLWGRGTLVTVSSASTKGPSV |
| | FPLAPSSKSTSGGTAALGCLVKDYFPEPVTVSWNSGALTSGVHTF |
| | PAVLQSSGLYSLSSVVTVPSSSLGTQTYICNVNHKPSNTKVDKRV |
| | EPKSCDKTHTCPPCPAPELLGGPSVFLFPPKPKDTLMISRTPEVT |
| | CVVVDVSHEDPEVKFNWYVDGVEVHNAKTKPREEQYNSTYRVVSV |
| | LTVLHQDWLNGKEYKCKVSNKALPAPIEKTISKAKGQPREPQVYT |
| | LPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK |

1. A process for the production of a recombinant polypeptide in a fed batch cell culture, comprising the steps of:
a. culturing mammalian cells in a cell culture medium comprising a base medium and one or more feed media, wherein the base medium comprises a concentration of cys equivalents of about 0.3 g/L and wherein the feed media comprises a concentration of cys equivalents of less than about 0.8 g/L, and wherein the concentration of cumulative cys equivalents in the cell culture media are less than about 0.4 g/L;
b. expressing the recombinant polypeptide and
c. recovering the polypeptide from the culture medium, wherein the recombinant polypeptide is an antibody.

2. The process according to claim 1, wherein the base medium comprises no added cysteine and the feed media comprise a concentration of cysteine of about 0.66 g/L.

3. The process according to claim 1, wherein the base medium comprises no added cysteine and the feed media comprise a concentration of cysteine of about 0.33 g/L.

4. The process according to claim 1, wherein the base medium comprises no added cysteine and the feed media comprise no cysteine.

5. (canceled)

6. The process according to claim 1, wherein the antibody is secukinumab.

7. The process according to claim 1, wherein the mammalian cells are selected from the group consisting of CHO cells, HEK cells and SP2/0 cells.

8. The process according to claim 1, comprising a downstream processing step of selective reduction, wherein the antibody is incubated with at least one reducing agent in a system to form a reducing mixture.

9. (canceled)

10. The process according to claim 1, wherein the antibody comprises at least one disulfide bond and at least one free cysteine.

11. (canceled)

12. The process according to claim 10, wherein the antibody is secukinumab.

13. The process according to claim 1, wherein a population of recombinant antibody polypeptides recovered from the culture medium comprises at least about a 10% higher level of reduced free cysteine as compared to a population of recombinant antibody polypeptides recovered from control culture media, the control culture media comprising a control base medium having a concentration of cys equivalents of greater than about 0.4 g/L and/or control feed media comprising a concentration of cys equivalents of greater than about 0.9 g/L, and/or wherein the concentration of cumulative cys equivalents in the control cell culture are greater than about 0.4 g/L.

14. The process according to claim 1, wherein the process comprises producing a higher yield, in mg of recombinant antibody polypeptide per L of culture media, of recombinant antibody polypeptide as compared to a control process comprising culturing mammalian cells in a control cell culture media comprising a control base medium and one or more control feed media, wherein the control base medium comprises a concentration of cys equivalents of greater than about 0.4 g/L and/or wherein the control feed media comprises a concentration of cys equivalents of greater than about 0.9 g/L, and/or wherein the concentration of cumulative cys equivalents in the control cell culture are greater than about 0.4 g/L.

15. The process according to claim 13, wherein the process comprises producing a population of recombinant antibody polypeptides having at least 61% reduced free cysteine as assayed from spent culture media.

16. A process for the production of a recombinant polypeptide by mammalian cell culture, comprising the steps of:
a. providing a culture comprising a cell culture medium and mammalian cells selected from the group consisting of CHO cells, HEK cells and SP2/0 cells, wherein the culture medium comprises a concentration of cys equivalents of about 0.3 g/L;
b. culturing mammalian cells;
c. exchanging a portion of the cell culture medium in the culture with fresh cell culture medium by perfusion, wherein the fresh cell culture medium comprises a concentration of cys equivalents of about 0.3 g/L, and/or wherein the concentration of cumulative cys equivalents added to the culture are less than about 7 g/L, or less than about 0.4 g/L per day;
d. expressing the recombinant polypeptide and
e. recovering the polypeptide from the culture,
wherein the recombinant polypeptide is an antibody.

17. The process according to claim 16, wherein the fresh culture medium comprises no added cysteine.

18. The process according to claim 16, wherein the process comprises exchanging at least 50% of the cell culture media with fresh cell culture medium by perfusion per day of culturing.

19. The process according to claim 16, wherein the recombinant antibody polypeptide comprises at least one free cysteine and at least one disulfide bond.

20. (canceled)

21. The process according to claim 16, wherein the recombinant antibody polypeptide is secukinumab.

22. The process according to claim 16, wherein a population of recombinant antibody polypeptides recovered from the culture medium comprises at least about a 10% higher level of reduced free cysteine as compared to a population of recombinant antibody polypeptides recovered from a control culture media, the control culture media comprising a concentration of cys equivalents of greater than about 0.4 g/L and/or wherein the concentration of cumulative cys equivalents added to the control cell culture are greater than about 7 g/L, and/or greater than about 0.4 g/L per day.

23. The process according to claim 16, wherein the process comprises producing a higher yield, in mg of recombinant antibody polypeptide per L of culture media, of recombinant antibody polypeptide as compared to a control process comprising culturing mammalian cells in a control cell culture medium, wherein the control cell culture medium comprises a concentration of cys equivalents of greater than about 0.4 g/L, and/or wherein the concentration of cumulative cys equivalents in the control cell culture media are greater than about 7 g/L, and/or greater than about 0.4 g/L per day.

24. The process according to claim 1, wherein the process further comprises covalently modifying the reduced free cysteine with a linker, a label, or a drug.


SEQUENCE LISTING (15 sequences)

SEQ ID NO: 1 - protein, 5 aa, Artificial Sequence
  HCDR1 Kabat
NYWMN

SEQ ID NO: 2 - protein, 17 aa, Artificial Sequence
  HCDR2 Kabat
AINQDGSEKYYVGSVKG

SEQ ID NO: 3 - protein, 18 aa, Artificial Sequence
  HCDR3 Kabat
DYYDILTDYYIHYWYFDL

SEQ ID NO: 4 - protein, 12 aa, Artificial Sequence
  LCDR1
RASQSVSSSYLA

SEQ ID NO: 5 - protein, 7 aa, Artificial Sequence
  LCDR2
GASSRAT

SEQ ID NO: 6 - protein, 9 aa, Artificial Sequence
  LCDR3
QQYGSSPCT

SEQ ID NO: 7 - DNA, 381 bp, Artificial Sequence
  VH DNA
gaggtgcagttggtggagtctgggggaggcttggtccagcctggggggtccctgagactctcctgtgcagcctctggattcacctttagtaactattggatgaactgggtccgccaggctccagggaaagggctggagtgggtggccgccataaaccaagatggaagtgagaaatactatgtgggctctgtgaagggccgattcaccatctccagagacaacgccaagaactcactgtatctgcaaatgaacagcctgagagtcgaggacacggctgtgtattactgtgtgagggactattacgatattttgaccgattattacatccactattggtacttcgatctctggggccgtggcaccctggtcactgtctcctca

SEQ ID NO: 8 - protein, 127 aa, Artificial Sequence [sequence omitted]

SEQ ID NO: 9 - DNA, 327 bp, Artificial Sequence
  VL DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcaccttcggccaagggacacgactggagattaaacga

SEQ ID NO: 10 - protein, 109 aa, Artificial Sequence
  VL amino acid
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 11 - protein, 10 aa, Artificial Sequence
  HCDR1-x Chothia
GFTFSNYWMN

SEQ ID NO: 12 - protein, 11 aa, Artificial Sequence
  HCDR2-x Chothia
AINQDGSEKYY

SEQ ID NO: 13 - protein, 23 aa, Artificial Sequence
  HCDR3-x Chothia
CVRDYYDILTDYYIHYWYFDLWG

SEQ ID NO: 14 - protein, 215 aa, Artificial Sequence
  Light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 457 aa, Artificial Sequence
  Heavy chain
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK